(19) United States
(12) Patent Application Publication
(10) Pub. No.: US 2026/0207253 A1
(43) Pub. Date: Jul. 23, 2026

(54) IRRIGATED BALLOON CATHETER FOR THE ABLATION OF TISSUE

(71) Applicant: Stockert GmbH, Freiburg im Breisgau (DE)

(72) Inventors: Frank Rehberger, Freiburg im Breisgau (DE); David Stork, Freiburg (DE); Colin Corcoran, Schallstadt (DE)

(21) Appl. No.: 19/453,452

(22) Filed: Jan. 20, 2026

(30) Foreign Application Priority Data
Jan. 20, 2025 (DE) 10 2025 101 828.6

(51) Int. Cl. A61B 18/14; A61B 18/00; A61M 25/10

(52) U.S. Cl. CPC A61B 18/1492; A61B 2018/00178; A61B 2018/00238; A61B 2018/00577; A61B 2018/00863; A61B 2018/1472; A61M 2025/105

(57) ABSTRACT

An application device, in particular a catheter device, a system with such a device for the ablation of tissue and a method for the ablation of tissue.

### CROSS-REFERENCE TO RELATED APPLICATIONS

This application claims priority to German Patent Application Serial No. DE 102025101828.6 filed January 20, 2025.

### BACKGROUND

### Field

The invention relates generally to an application device, in particular a catheter device, a system with such a device for the ablation of tissue and a method for the ablation of tissue.

### Discussion of Related Art

The treatment of tissue by means of electrical energy, such as high-frequency (HF) currents or pulsed electric fields, for example, is an established clinical technique. In HF ablation, an applicator is introduced into the tissue and a high-frequency current is generated. In pulsed field ablation (PFA), short electric high-voltage pulses and high electric field strengths associated with these, which act on the tissue, are utilised. This application method is categorised as a non-thermal procedure, as it is based on the delivery of short pulses with a high voltage amplitude, which produce a locally strong electric field in the range of up to several thousand volts per centimetre between active electrode pairs. This field strength leads to the temporary formation of pores in the cell membranes. If the electric field exceeds a certain threshold value, which is necessary for the formation of pores in the lipid bilayers of the cell membranes, and if the tissue is exposed to this field over a critical period, the cells die due to apoptosis (a form of programmed cell death).

To treat cardiac arrhythmias such as e.g. atrial fibrillation, applicators are used in this case that are often catheters with electrodes on so-called cup structures, balloons or ring electrodes mounted on a tube.

The present invention falls in the scope of minimally invasive medical procedures, with a special focus on ablation technology. In particular, it is concentrated on electrical energy delivery via HF ablation and irreversible electroporation (IRE), so-called pulsed field ablation (PFA) of tissue. IRE is an advanced method that is used in interventional medicine for the targeted treatment of tissue. This technique represents a significant advance in medical therapy and is already used specifically for the ablation of heart tissue, taking particular account of tissue selectivity, reduced treatment times and the minimisation of risks of conventional treatment methods.

From the prior art, US 11690671 B1 is known, which relates to a tissue ablation catheter with an insulator between the inner and outer electrode. The arrangement discloses electrodes offset at a distal end. A material between inner and outer electrode is an insulator. This catheter configuration generates an electric field, which bends around the tip of the catheter.

A requirement exists for an improved application device, in particular catheter device, and for an associated system and method for the ablation of tissue.

### SUMMARY

According to a first aspect, an application device is proposed. The application device can be formed in particular as a catheter device. The application device, in particular catheter device, is often designated herein only as a device in short. The device can serve in particular for use in surgery.

The (application) device has a shaft, which is adapted to receive at least one electrically conductive fluid, in particular to convey it from a proximal end of the shaft to a distal end of the shaft. The device has at least one first electrode, which is arranged on a first section of the shaft. The device has at least one first counter electrode. The device has a first electric line, which is connected to the at least one first electrode and the at least one first counter electrode. The first electric line is adapted to transmit an electrical signal to be received to the at least one first electrode and the at least one first counter electrode. The device has at least one first, in particular electrically insulating, membrane. The at least one first membrane is adapted to enclose the first section of the shaft, to end with the shaft in a fluid-tight, in particular liquid-tight, manner and to form a first interior space between the at least one first membrane and the first section of the shaft. The first section of the shaft is adapted to convey the at least one electrically conductive fluid into the interior space. A first wall of the at least one first membrane is adapted to be at least partially permeable to fluid, in particular permeable to liquid.

One advantage of the device is that an electrical connection to the fluid is brought about, so that the at least partially fluid-permeable membrane focuses a voltage pulse emitted at a/the (e.g. at least one first) electrode and produces so to speak “virtual electrodes” at locations at which the electrically conductive fluid emerges from the membrane. At the same time, the thermal effect on the tissue is targetedly reduced by the emerging fluid. The complexity is reduced at the same time, as only one electrical connection is required.

This has the advantage that on the one hand, thermal effects can be reduced by electrically conductive fluid, for example an irrigation solution, and at the same time the tissue contact and an occlusion associated with this can be ensured by the membrane.

The electrically conductive fluid can have a first and/or a second electrically conductive fluid or be formed as a first and/or a second electrically conductive fluid. The shaft of the device can be adapted to receive the first and/or second electrically conductive fluid. The first section of the shaft can be adapted to convey the first and/or the second electrically conductive fluid into the interior space.

The at least one first membrane, in particular the first wall, can have at least one first perforation, from which the, in particular the first and/or second, electrically conductive fluid can emerge.

The first section can be arranged between the distal end and the proximal end.

The device can have at least one second electrode. The at least one first and/or second electrode can be arranged on a second and/or the first section of the shaft and be connected to the first electric line.

The second section can be arranged between the first section and the proximal end.

The device can have at least one second, in particular electrically insulating, membrane. The at least one second membrane can be adapted to enclose the second section of the shaft, to end with the shaft in a fluid-tight, in particular liquid-tight manner, and to form a second interior space between the at least one second membrane and the second section of the shaft.

The second section of the shaft can be adapted to convey a/the, in particular the first and/or second, electrically conductive fluid into the second interior space. A second wall of the at least one second membrane can be adapted to be permeable to fluid, in particular permeable to liquid.

The at least one second membrane, in particular the second wall, can have at least one second perforation from which the, in particular the first and/or second, electrically conductive fluid can emerge.

The first and/or the second interior space can have at least one first fluid chamber and/or one second fluid chamber. The at least one first and/or second fluid chamber can be adapted to enclose the first and/or second section of the shaft in a fluid-tight manner/to be connected to the first and/or second section of the shaft in a fluid-tight manner. The at least one first fluid chamber can be adapted to convey the or a first electrically conductive fluid to at least one first partial surface of an interior surface of the first and/or second membrane. The at least one second fluid chamber can be adapted to convey the or a second electrically conductive fluid to at least one second partial surface of the interior surface of the first and/or second membrane. The first and/or second fluid chamber can include the at least one first and/or the at least one second electrode. The at least one first and/or second electrode can be arranged on a first and/or second part of the first or the second section of the shaft.

The at least one first fluid chamber can be connected to the at least one first partial surface of an interior surface of the at least one first and/or the at least one second membrane in a fluid-tight manner. The at least one second fluid chamber can be connected to the at least one second partial surface of the interior surface of the at least one first and/or the at least one second membrane in a fluid-tight manner.

The at least one first and/or second fluid chamber can occupy/cover any angular area (about a longitudinal axis of the first and/or second membrane) between 45^{o} and 360^{o}. The at least one first and/or second fluid chamber can each occupy/cover any angular area, so that a total angular area of both fluid chambers of between 45^{o} and 360^{o} results (e.g. first angular area 90^{o}, second angular area 180^{o}, total angular area 270^{o}).

The formation of one or more fluid chambers has the advantage that only certain points of the tissue receive an application by means of the energy. An unnecessary injection of energy into tissue that is not to be ablated is thus prevented.

The at least one first, the at least one second electrode, the at least one first, second, third (see below) counter electrode and/or the membrane(s), in particular the at least one first and/or second perforation, can have a bioresorbable material, which is soluble in particular in bodily fluids, or can be covered with a bioresorbable material, which is soluble in particular in bodily fluids, or a corresponding material layer.

The at least one first membrane can be arranged between the distal end (of the shaft) and the proximal end (of the shaft). The at least one second membrane can be arranged between the at least one first membrane and the proximal end of the shaft. The at least one first and/or the at least one second membrane can be formed as a balloon.

The device can further have at least one containment structure. The at least one containment structure can enclose a second part of the shaft in a fluid-tight, in particular liquid-tight, manner. The at least one containment structure can be adapted to confine or impede the electrically conductive fluid emerging from the first wall, in particular from the at least one first perforation, in a propagation direction, and/or to be displaceable or displaced along the shaft. The at least one containment structure can be arranged between the at least one first membrane and the proximal end. The at least one containment structure can be adapted to end with the shaft in a fluid-tight manner.

The device can have a discharge opening/drainage opening. The discharge opening can be arranged on the shaft or integrated into the shaft. The discharge opening can be arranged on the shaft between the at least one first membrane and the at least one containment structure or between the at least one first membrane and the at least one second membrane. The discharge opening can be adapted to discharge the electrically conductive fluid that has emerged from the first wall, in particular from the at least one first perforation, and/or from the second wall, in particular from the at least one second perforation, e.g. into the shaft. The shaft can be adapted to convey the discharged electrically conductive fluid e.g. to the proximal end or via the proximal end out of the shaft. The shaft can have a discharge line arranged in the shaft and connected to the discharge opening.

At least one first, at least one second and/or at least one third counter electrode can be provided. For example, at least one first, at least one second and/or at least one third counter electrode can be provided between: the distal end and the at least one first membrane, or between the at least one first membrane and the proximal end, or between the at least one first and the at least one second membrane, or between the at least one second membrane and the proximal end, or arranged between the at least one first membrane and the at least one containment structure. The at least one first, at least one second and/or at least one third counter electrode can be arranged adjacent to one another or separate from one another. The at least one first and/or at least one second and/or at least one third counter electrode can be formed as a body electrode.

The at least one first and/or the at least one second perforation can be formed as at least one perforation line. The at least one perforation line can be oriented along a longitudinal axis of the first and/or second membrane, in particular starting from a middle/centric membrane periphery or starting from a first or second end of the first and/or second membrane, in a longitudinal axis direction to a second or first end of the first and/or second membrane.

The at least one first and/or the at least one second perforation can be formed as a plurality of perforation lines, which are each oriented along a longitudinal axis of the first and/or second membrane, in particular starting from a middle/centric periphery or starting from a first or second end of the first and/or second membrane, in a longitudinal axis direction to a second and/or first end of the first and/or second membrane. The at least one first and/or the at least one second perforation can be formed as at least one perforation line or as a plurality of perforation lines or as a plurality of perforations, which can be arranged or distributed along or across an in particular middle/centric periphery of the first and/or second membrane.

The at least one first and/or second partial surface of the interior can (e.g. exclusively) correspond to the at least one first and/or to the at least one second perforation. The at least one first and/or second partial surface of the interior can (e.g. exclusively) correspond to the at least one perforation line. The at least one part of the interior can (e.g. exclusively) correspond to one or more perforation line(s) of the plurality of perforation lines.

The at least one first and/or second partial surface of the interior surface can correspond to a complete area of the interior surface, which results/extends from the first end of the first or second membrane, in particular along the longitudinal axis of the first and/or second membrane, towards the middle/centric periphery. The at least one first and/or second partial surface of the interior can correspond to an area of the interior surface, which results/extends starting from the first end of the first or second membrane to the second end of the corresponding membrane.

The first and/or the second section can have a filling opening, wherein the first and/or the second interior space can be filled or is filled with the electrically conductive fluid via the proximal end. The first and/or the second section can have a vent opening, wherein the first and/or the second interior space can be vented or is vented via the proximal end.

The shaft can have a vent line arranged in the shaft and connected to the vent opening. The shaft can have a first and/or second filling line arranged in the shaft and connected to the filling opening.

The first and/or the second membrane can, in particular each, have a first end and/or a second end. In a device longitudinal axis that is vertically oriented (outside the body), the interior space can be vented when the interior space is filled with the electrically conductive liquid. The interior space can be vented when the device is oriented/held vertically and the interior space is filled with an electrically conductive liquid.

The device, the first section and/or the second section and/or an exterior of the first and/or second membrane can have a fluid pressure measurement sensor and/or a fluid flow measurement sensor. The fluid pressure measurement sensor and/or fluid flow measurement sensor can each be adapted to provide measuring data. The measuring data can be read via a second electric line provided or arranged in the shaft.

This has the advantage that a pressure measurement of the fluid can be measured at different areas of the device when it is located in a lumen of a patient, whereby a more accurate statement about the degree of occlusion and the load for the patient can be determined. A fluid supply can be adapted via the degree of occlusion.

The first and/or the second electric line can be electrically insulated from the electrically conductive fluid.

The proximal end of the shaft can have an operating unit. The operating unit can have an inflow and outflow connection. The inflow and outflow connection can be adapted to convey an electrically conductive fluid to be received into the shaft, in particular to fill the interior space/interior spaces with a/the electrically conductive fluid to be received. The inflow and outflow connection can be adapted to convey emerged and discharged electrically conductive fluid out of the shaft.

The operating unit can have an electrical connection connected to the first and/or the second electric line. The electrical connection can be adapted to transmit electrical signals to be received to the first electric line and/or to receive and transmit onwards electrical signals, in particular measuring data from the fluid pressure measurement sensor and/or fluid flow measurement sensor, by means of the second electric line.

The operating unit can have an introducer opening, in particular formed as a haemostatic valve. The introducer opening can be adapted to receive a guide wire. The guide wire can be routed to or to and out of an exit opening at the distal end of the shaft.

According to a second aspect, a system for the ablation of tissue is proposed. The system has at least one device according to the first aspect, at least one signal generator arrangement connected or connectable to the device, at least one control and evaluation unit connected or connectable to the device and/or signal generator arrangement, and/or at least one fluid supply unit connected or connectable to the device and/or control and evaluation unit.

The fluid supply unit can be adapted to provide at least one or a first and a second electrically conductive fluid.

The control and evaluation unit can be adapted to set a volume flow of the fluid supply unit, in particular following a setting by a user. The fluid supply unit can be adapted to provide the (or the first and/or second) electrically conductive fluid according to the setting and to measure a fluid pressure and/or fluid flow by means of the fluid pressure measurement sensor and/or the fluid flow measurement sensor.

This has the advantage that on the one hand, thermal effects are reduced by an electrically conductive fluid, for example an irrigation solution, and at the same time the tissue contact and an occlusion connected thereto can be ensured by the membrane(s) and dynamic pressure also quantified.

The control and evaluation unit can be adapted to determine and output from the measured fluid pressure and/or fluid flow a measure for the positive fit between the membrane(s), in particular the at least one perforation, and the tissue to be ablated. The measure for the positive fit can be indicated by a coverage in percent, for example 80% coverage. The measure for the positive fit can be indicated in millilitres per minute, in particular millilitres per minute, of a recirculation, and/or in coverage per percent.

The (the first and/or the second) electrically conductive fluid can be formed as an electrically conductive liquid and/or as an electrically conductive liquid active substance, in particular can have a vasoactive substance or be formed as such, in particular (liquid) histamine, which on contact with tissue changes its electrical conductivity, in particular increases or reduces it. The vasoactive substance can act in a vasodilatory or vasoconstrictive manner.

This has the advantage that the tissue section to be ablated is in contact with an active substance that increases the electrical conductivity. The ablation is thus configured more effectively, wherein a tissue section that is not to be ablated and is adjacent to a tissue section to be ablated can have its electrical conductivity reduced by conductivity-reducing substances and the energy input during ablation can be reduced thereby.

The electrically conductive fluid, in particular the vasoactive substance, can have bradykinin, serotonin, betahistine, acetylcholine, dopamine, noradrenaline, Angiotensin II, Angiotensin II and/or catecholamine or be formed as such or as a combination as such.

The signal generator arrangement can have a first signal generator for generating a radiofrequency (RF) signal and a second signal generator for generating a signal for pulsed field ablation.

The control and evaluation unit can be adapted to activate the first signal generator and/or the second signal generator to deliver a signal.

According to a third aspect, a method is proposed for the ablation of tissue or for the delivery of electrical energy to tissue, in particular ablation, in particular for irreversible ablation. The method comprises provision of a system. The system has an application device according to the first aspect.

The system has a signal generator arrangement connected or connectable to the application device. The system has a control and evaluation unit connected or connectable to the application device and/or signal generator arrangement. The system has a fluid supply unit connected or connectable to the application device and/or control and evaluation unit, which fluid supply unit is adapted to provide an electrically conductive fluid, e.g. a vasoactive substance.

The method can comprise introduction of the application device into a lumen of a patient as far as a tissue section to be ablated. The method can comprise setting the control and evaluation unit to set the volume flow or setting a volume flow at the control and evaluation unit. The method comprises activation of the control and evaluation unit to generate an electrical signal.

The electrically conductive fluid can have a vasoactive substance. The method can comprise fine-positioning of the application device relative to the tissue section to be ablated. The method can comprise delivery of an electrical signal. The method can comprise delivery of a vasoactive substance. The method can comprise removal of the application device from the lumen of the patient.

The method can comprise contacting of the application device with the tissue. The method can comprise application of the electrically conductive fluid, e.g. the vasoactive substance. Following application, the activation of the control and evaluation unit to generate an electrical signal can take place directly.

The application device can be adapted to transmit an electrical signal to be received and a vasoactive, in particular liquid, substance to be received, in particular histamine, to/into the tissue.

According to a fourth aspect, a method for ablation, in particular irreversible ablation, of tissue or for the delivery of electrical energy to tissue is proposed. The method comprises provision of a system. The system has a device according to the first aspect, a signal generator arrangement connected or connectable to the device, a control and evaluation unit connected or connectable to the device and/or signal generator arrangement and a fluid supply unit connected or connectable to the device and/or control and evaluation unit.

The application device is adapted to transmit an electrical signal to be received and a vasoactive, in particular liquid, substance to be received, in particular histamine, to/into the tissue. The fluid supply unit is adapted to provide the vasoactive substance.

The method comprises contacting of the application device with the tissue. The method comprises application of the vasoactive substance. The method comprises activation of the control and evaluation unit to generate an electrical signal.

The method can comprise setting the control and evaluation device to set the volume flow. The method can, following application, comprise direct activation of the control and evaluation unit to generate an electrical signal.

The method can comprise introduction of the device into a lumen of a patient as far as a tissue section to be ablated. The method can comprise setting a volume flow at the fluid supply unit or control and evaluation unit. The method can comprise activation of the control and evaluation unit to generate an electrical signal.

Further features, properties, advantages and possible modifications become clear to an expert based on the descriptions below, in which reference is made to the enclosed drawings.

### BRIEF DESCRIPTION OF THE DRAWINGS

FIG. 1 shows a schematic representation of a variant of an embodiment with a membrane.

FIG. 1a shows a schematic representation of a variant of an embodiment with a membrane and two fluid chambers.

FIG. 1b shows a schematic representation of a variant of an embodiment with a membrane and one fluid chamber.

FIG. 2 shows a schematic representation of another variant of an embodiment with two membranes.

FIG. 3 shows a schematic representation of a variant of an embodiment with an operating unit.

FIG. 4 shows a schematic representation of a variant of an embodiment with drainage openings.

FIG. 5 shows a schematic representation of a variant of an embodiment with a containment element.

FIG. 6 shows a schematic representation of a variant of an embodiment with a field pattern during ablation.

FIG. 7 shows a method for delivering electrical energy to tissue.

FIG. 8 shows a method for irreversible ablation.

### DETAILED DESCRIPTION OF THE EMBODIMENTS

In the figures described below, the application device is formed by way of example as a catheter device in each case.

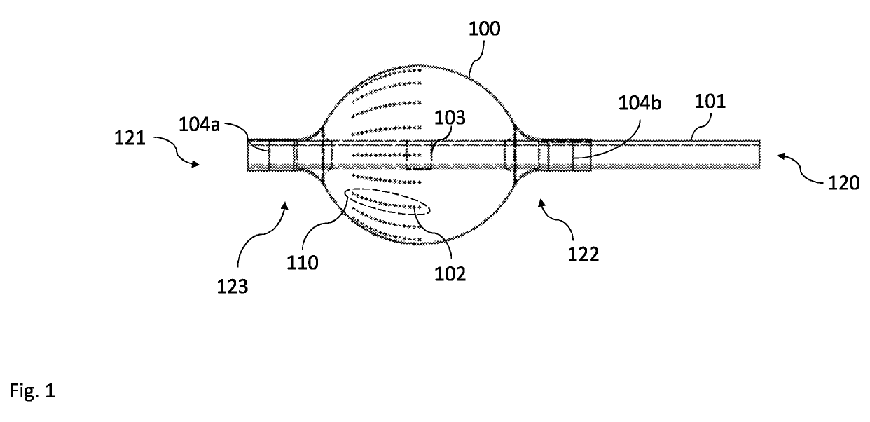

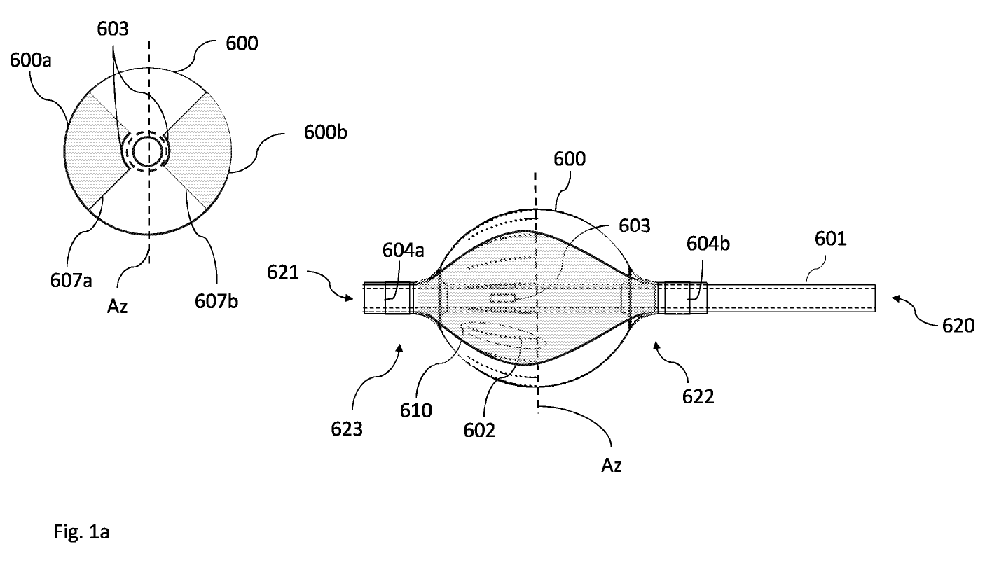

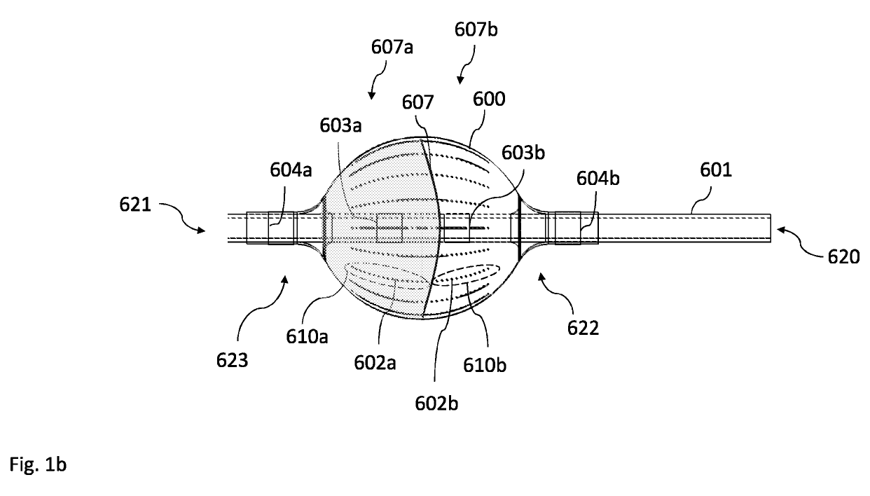

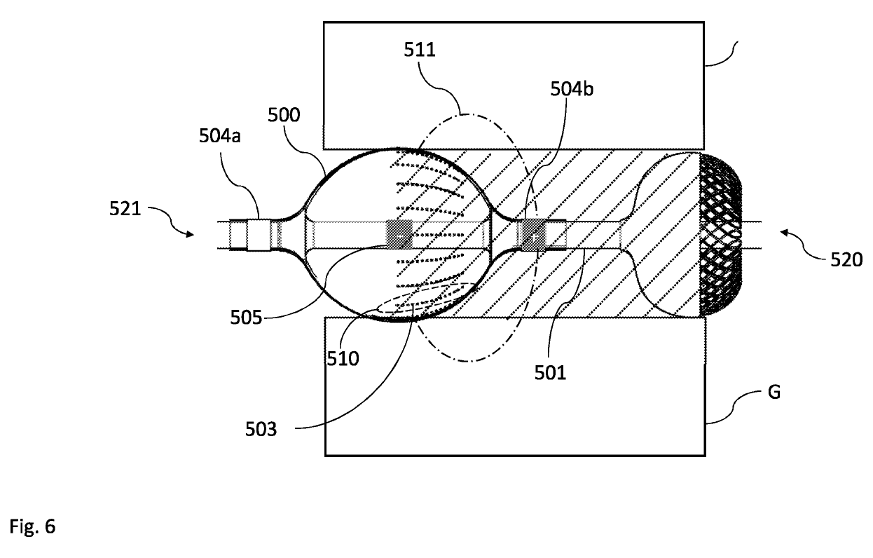

FIG. 1 shows a schematic representation of a variant of an embodiment of an application device, which is formed in the example shown as a catheter device and is therefore also described as such, with a membrane 100. The variant depicted has a shaft 101, which has in turn a proximal end 120 and a distal end 121. Arranged on a first section of the shaft 101 is a first electrode 103. The first electrode 103 is enclosed by a membrane 100 in a fluid-tight manner. Expressed differently, a first end 122 and a second end 123 of the membrane are connected to the shaft 101 in a fluid-tight manner. If the membrane 100 encloses the first section of the shaft 101, a first interior space is formed between the membrane 100 and the shaft 101. The membrane 100 has at least a single perforation 102, which is formed in the variant depicted as a plurality of perforation lines. One of the plurality of perforation lines is marked by 110 and a dashed line. Each of the plurality of perforation lines is oriented along a longitudinal axis of the membrane 100. Each of the perforation lines begins on the centric periphery of the membrane 100 and ends in the variant depicted at the second end 123 of the membrane 100. Apart from the first electrode 103, two other counter electrodes 104a, 104b are arranged on the shaft 101 outside the first interior space. The proximal end 120 of the shaft 101 is adapted to receive an electrically conductive fluid and to convey it to the distal end 121 of the shaft 101. The first section of the shaft 101 is adapted to convey the electrically conductive fluid into the first interior space. The first interior space is filled with the electrically conductive fluid. The electrically conductive fluid emerges from the plurality of perforation lines. If an electrical signal is applied between the first electrode 103 and the counter electrode 104a or 104b, improved signal guidance into the tissue to be ablated takes place on account of the electrically conductive fluid that has emerged, which is explained in greater detail in FIG. 6.

FIG. 1a shows a schematic representation of a variant of an embodiment with a membrane 600 and two fluid chambers 607a, 607b. The variant depicted has a shaft 601, which in turn has a proximal end 620 and a distal end 621. Arranged on one part of a first section of the shaft 601 in each case is a single electrode 603. The electrodes 603 are enclosed by a membrane 600 in a fluid-tight manner. Expressed differently, a first end 622 and a second end 623 of the membrane are connected to the shaft 601 in a fluid-tight manner. If the membrane 600 encloses the first section of the shaft 601, a first interior space is formed between the membrane 600 and the shaft 601. The membrane 600 has at least a single perforation 602, which is formed in the variant depicted as a plurality of perforation lines. One of the plurality of perforation lines is marked by 610 and a dashed line. Each of the plurality of perforation lines is oriented along a longitudinal axis of the membrane 600. Each of the perforation lines begins at the centric periphery of the membrane 600 and ends in the variant depicted at the second end 623 of the membrane 600. Apart from the electrodes 603, two further electrodes 604a, 604b are arranged on the shaft 601 outside the first interior space. The proximal end 620 of the shaft 601 is adapted to receive a first electrically conductive fluid and to convey it to the distal end 621 of the shaft 601.

The interior space has a first and a second fluid chamber 607a, 607b. Each of the fluid chambers 607a, 607b is connected to respectively one part of the first section of the shaft 601 in a fluid-tight manner, on which part one of the electrodes 603 is arranged. The first fluid chamber 607a is connected in a fluid-tight manner to a first partial surface 600a of an interior surface of the membrane 600. The second fluid chamber 607b is connected in a fluid-tight manner to a second partial surface 600b of the interior surface of the membrane 600. Both fluid chambers 607a, 607b are arranged opposite one another. Expressed differently, the first electrical fluid is flushed into the fluid chambers 607a, 607b via the first section of the shaft 601 and the fluid chambers 607a, 607b convey the electrically conductive fluid exclusively to their corresponding partial surfaces.

The first and second partial surface 600a, 600b correspond to an area of the interior surface of the membrane 600 that extends from the first end 622 of the membrane 600 to the second end 623 of the membrane 600. Not all perforation lines of the membrane 600 are thereby irrigated with the electrically conductive fluid. This is shown once again in the sectional representation at the top left in FIG. 1a. It is to be recognised that the two fluid chambers 607a, 607b are arranged to each side of a vertical axis Az. The partial surfaces (not marked) of the membrane 600 that cross the vertical axis Az are free of contact with the fluid chambers 607a, 607b. Thus in the event of an ablation application, only the tissue sections that correspond to the two partial surfaces 600a, 600b of the membrane 600 are treated.

In an alternative variant of the embodiment, the two fluid chambers 607a, 607b can be arranged anywhere along the interior surface of the membrane 600 depending on the purpose of use. For example, both fluid chambers 607a, 607b can immediately adjoin one another. In the variant shown, the two fluid chambers each cover an angular area of approximately 90^{o} (see sectional representation) and take an overall angular area of approximately 180^{o}. Alternatively, depending on the composition of the tissue to be ablated, each fluid chamber 607a, 607b can occupy any angular area, so that a total angular area of both fluid chambers 607a, 607b of between approximately 45^{o} and 360^{o} results (first angular area approximately 90^{o}, second angular area approximately 180^{o}, total angular area approximately 270^{o}).

Alternatively, one fluid chamber can be irrigated with a first electrically conductive fluid, while the second fluid chamber is irrigated with a second electrically conductive fluid.

Alternatively, one or two additional fluid chambers can be arranged in the interior space. The first and second fluid chamber can be irrigated with the first electrically conductive fluid, while the two additional fluid chambers are irrigated via the first shaft with a second electrically conductive fluid.

Alternatively, a fluid chamber can first be irrigated with a first electrically conductive fluid, and thereupon with a second electrically conductive fluid.

The first electrically conductive fluid is formed as an example as vasoactive liquid that increases electrical conductivity in particular, and the second electrical liquid is formed as an example as vasoactive liquid that reduces electrical conductivity in particular.

FIG. 1b shows a schematic representation of a variant of an embodiment with a membrane and a fluid chamber 607. The variant depicted has a shaft 601, which in turn has a proximal end 620 and a distal end 621. Arranged on respectively one part of a first section of the shaft 601 is a first electrode 603a and second electrode 603b. The first electrode 603a and the second electrode 603b are enclosed in a fluid-tight manner by a membrane 600. Expressed differently, a first end 622 and a second end 623 of the membrane 600 are connected to the shaft 601 in a fluid-tight manner. If the membrane 600 encloses the first section of the shaft 601, a first interior space is formed between the membrane 600 and the shaft 601. The membrane 600 has at least a first single perforation 602a, which is formed in the variant depicted as a plurality of perforation lines. One of the plurality of perforation lines is marked by 610a and a dashed line. Each of the plurality of perforation lines is oriented along a longitudinal axis of the membrane 600. Each of the perforation lines 610a begins at the centric periphery of the membrane 600 and ends in the variant depicted at the second end 623 of the membrane 600. Apart from the first and second electrode 603a, 603b, two further counter electrodes 604a, 604b are arranged outside the first interior space on the shaft 601. The proximal end 620 of the shaft 601 is adapted to receive a first electrically conductive fluid and to convey it to the distal end 621 of the shaft 601.

The interior space has a first and second fluid chamber 607a, 607b. These are formed by an in particular fluid-impermeable separating membrane 607 in the interior space. The separating membrane 607 in the interior space divides the interior space into two fluid chambers 607a, 607b of approximately equal size.

The pertinent fluid chamber 607a, 607b can be filled via the first and/or second part of the section. Here either one of the two or both fluid chambers 607a, 607b can be filled. In the variant depicted, the first fluid chamber 607a is irrigated as an example.

The first fluid chamber 607a is connected in a fluid-tight manner to the first part of the first section of the shaft 601, on which the first electrode 603a is arranged. The separating membrane 607 ends in a fluid-tight manner with the shaft and the inside of the membrane 600. Expressed differently, a first electrically conductive fluid is flushed into the first fluid chamber 607a via the first part of the section of the shaft 601 and the first fluid chamber 607a conveys the first electrically conductive fluid to the first partial surface.

Expressed differently, the first electrically conductive fluid is flushed into the first fluid chamber 607a via the first section of the shaft 601 and the fluid chamber 607a conveys the electrically conductive fluid exclusively to the first partial surface.

The first partial surface corresponds to a complete area of the interior surface of the membrane 600, which extends from the second end 623 of the membrane 600 to a centric periphery of the membrane 600. Expressed differently, the first fluid chamber 607a covers an angular area about the longitudinal axis of the membrane of 360^{o}.

Alternatively, the first partial surface can correspond to the plurality of perforation lines. Alternatively, the first partial surface can correspond to exclusively one perforation line of the plurality of perforation lines or to any (adjacent or next but one adjacent) selection of perforation lines of the plurality of perforation lines. Alternatively, an additional fluid chamber can be provided, which conveys the first or a second electrically conductive fluid to a second partial surface, in particular to another selection of perforation lines, or to a single perforation line that is not part of the first partial surface.

In one variant with a first and an additional fluid chamber, each fluid chamber can occupy any angular area about the longitudinal axis of the membrane, so that a total angular area of both fluid chambers of between 45^{o} and 360^{o} results (first angular area 180^{o}, second angular area 180^{o}, total angular area 360^{o}).

The additional fluid chamber can be irrigated in a variant of an embodiment via the first shaft with a second electrically conductive fluid, while the first fluid chamber is irrigated with the first electrically conductive fluid. The first electrically conductive fluid is formed as an example as a vasoactive liquid and the second electrical liquid is formed by way of example as a conductivity-reducing liquid.

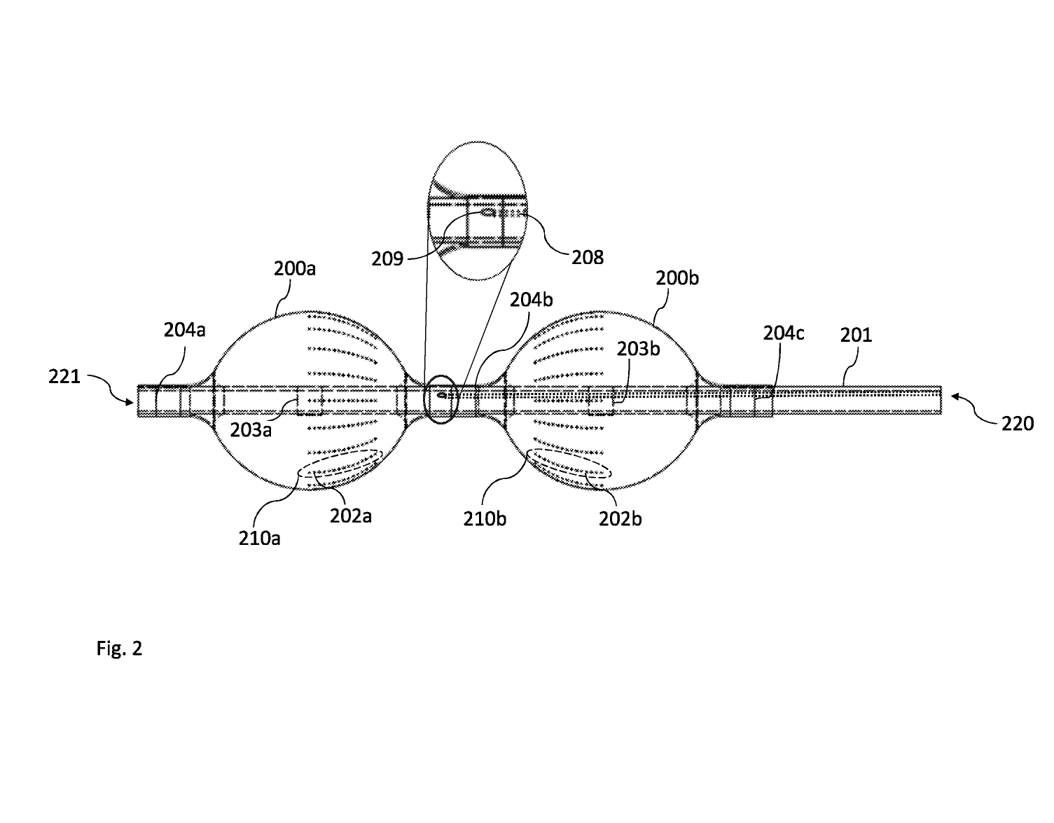

FIG. 2 shows a schematic representation of another variant of an embodiment of the catheter device with two membranes 200a, 200b. The variant depicted has a shaft 201, which has in turn a proximal end 220 and a distal end 221. Arranged on a first section of the shaft 201 is a first electrode 203a. The first section is enclosed in a fluid-tight manner by a membrane 200a. Expressed differently, a first end and a second end of the membrane 200a are connected to the shaft 201 in a fluid-tight manner. If the membrane 200a encloses the first section of the shaft 201, a first interior space is formed between the membrane 200a and the shaft 201. The membrane 200a has at least one single perforation 202a, which is formed in the variant depicted as a plurality of perforation lines. One of the plurality of perforation lines is marked by 210a and a dashed line. Each of the plurality of perforation lines is oriented along a longitudinal axis of the membrane 200a. Each of the perforation lines begins at the centric periphery of the membrane 200a and ends in the variant depicted at the first end of the membrane 200a.

A second electrode 203b is arranged on a second section of the shaft 201. The second section is enclosed by a membrane 200b in a fluid-tight manner. Expressed differently, a first end and a second end of the membrane 200b are connected to the shaft 201 in a fluid-tight manner. If the membrane 200b encloses the second section of the shaft 201, a second interior space is formed between the membrane 200b and the shaft 201. The membrane 200b has at least one single perforation 202b, which is formed in the variant depicted as a plurality of perforation lines. One of the plurality of perforation lines is marked by 210b and a dashed line. Each of the plurality of perforation lines is oriented along a longitudinal axis of the membrane 200b. Each of the perforation lines begins at the centric periphery of the membrane 200b and ends in the variant depicted at the second end of the membrane 200b.

Apart from the first electrode 203a and the second electrode 203b, three counter electrodes 204a, 204b, 204c are arranged on the shaft 201 outside the first and second interior space. The proximal end 220 of the shaft 201 is adapted to receive an electrically conductive fluid and to convey it to the distal end 221 of the shaft 201. The shaft 201 has a wall. In the first section of the shaft 201, the wall has a first filling opening (not depicted), via which the electrically conductive fluid is conveyed into the first interior space. The first interior space is filled with the electrically conductive fluid. In the second section of the shaft 201, the wall has a second filling opening (not depicted), via which the electrically conductive fluid is conveyed into the second interior space. The second interior space is filled with the electrically conductive fluid. The electrically conductive fluid emerges from the plurality of perforation lines of each membrane 200a, 200b.

In the variant depicted, when the device is guided into a lumen (not depicted), an intermediate space is formed between the first and second membrane 200a, 200b and the tissue wall (not depicted) bordering the device. If the electrically conductive fluid emerges from the plurality of perforation lines 202a, 202b, the intermediate space is filled with the electrically conductive fluid and is in contact with the tissue wall.

If an electrical signal is applied between the first electrode 203a and the counter electrode 204b, improved field line guidance takes place into the tissue to be ablated on account of the electrically conductive fluid that has emerged, which is explained in greater detail in FIG. 6.

To reduce the load for the lumen, a discharge opening or drainage opening 209 is provided on the shaft 201 in the variant depicted. The electrically conductive fluid is conveyed out of the intermediate space via the discharge opening 209 into the interior of the shaft 201. In the shaft, a discharge line 208 is provided, via which the electrically conductive fluid located in the intermediate space is conveyed to the proximal end 220 of the shaft 201.

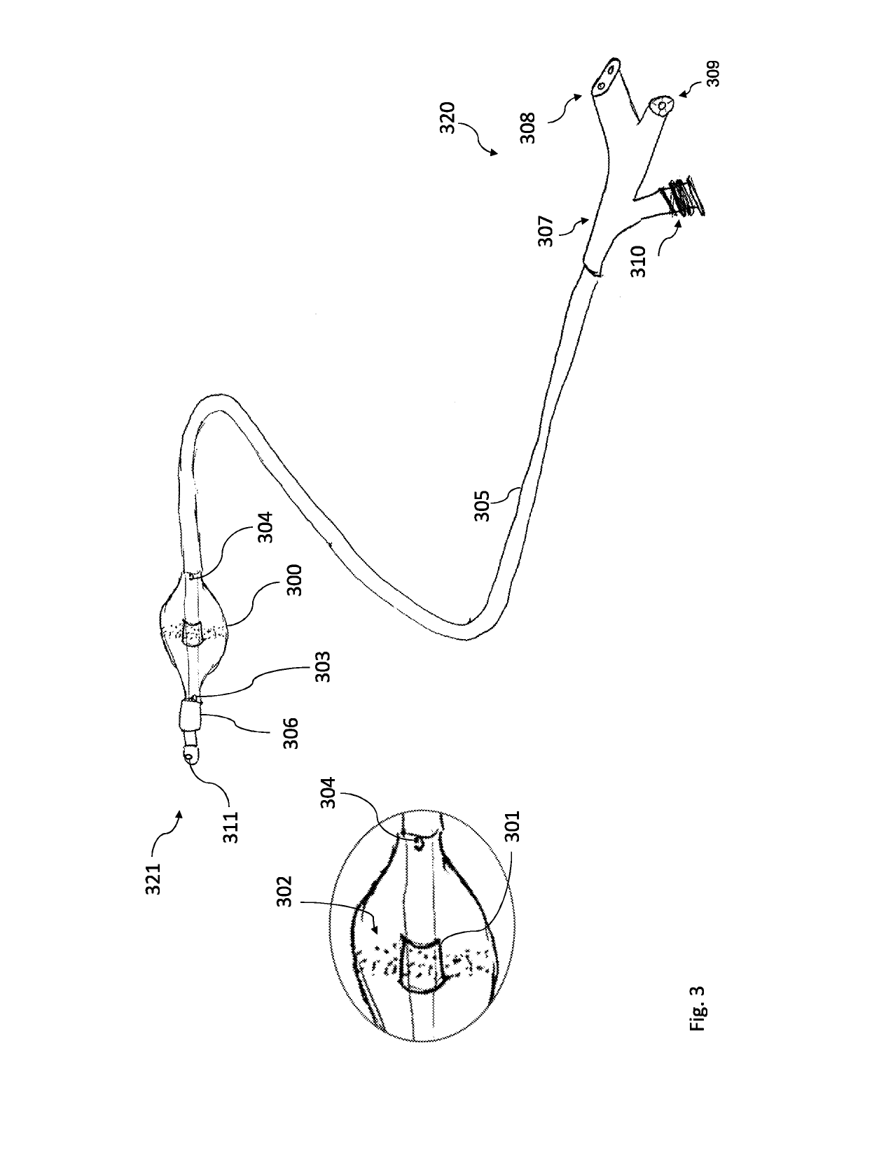

FIG. 3 shows a schematic representation of a variant of an embodiment with operating unit 307. The variant depicted has a shaft 305, which has in turn a proximal end 320 and a distal end 321. A first electrode 301 is arranged on a first section of the shaft 305. The first section is enclosed by a membrane 300 in a fluid-tight manner. Expressed differently, a first end and a second end of the membrane 300 are connected to the shaft 305 in a fluid-tight manner. If the membrane 300 encloses the first section of the shaft 305, a first interior space is formed between the membrane 300 and the shaft 305. The membrane 300 has at least one single perforation, which is formed in the variant depicted as a plurality of perforations 302. The plurality of perforations 302 is distributed along a middle periphery of the membrane 300. Apart from the first electrode 301, a counter electrode 306 is arranged outside the first interior space on the shaft 305.

The proximal end 320 of the shaft 305 has an operating unit 307 and an inflow and outflow connection 310, via which an electrically conductive fluid is conveyed into the shaft and to the membrane 300 in the variant depicted.

In the variant depicted, the operating unit 307 has an electrical connection 308, which is connected to an electric line (not shown) arranged in the shaft. The electric line is connected to the first electrode 301 and the one counter electrode 306.

The operating unit 307 has an introducer opening 309 formed as a haemostatic valve, via which a guide wire can be guided into the shaft 305 and as far as the distal end 321. The distal end 321 has an outlet opening 311, from which the guide wire can be conveyed out of the distal end 321.

Arranged on the first section of the shaft 305 apart from the first electrode is at least one filling opening 304, which is connected to the interior of the shaft 305 and from which the electrically conductive fluid gets into the first interior space and fills it. Arranged on the first section of the shaft 305 apart from the first electrode is at least one vent opening 303, which is connected to the interior of the shaft 305 and conveys air out of the first interior space when the first interior space is filled with the electrically conductive fluid.

The electrically conductive fluid emerges from the plurality of perforations 302.

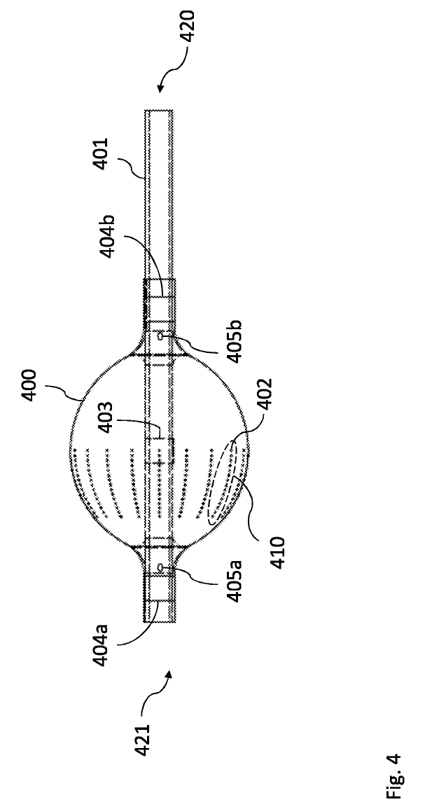

FIG. 4 shows a schematic representation of a variant of an embodiment with a vent opening 405a and a filling opening 405b. The variant depicted shows a shaft 401, which has in turn a proximal end 420 and a distal end 421. A first electrode 403 is arranged on a first section of the shaft 401. The first section is enclosed by a membrane 400 in a fluid-tight manner. Expressed differently, a first end 122 and a second end 123 of the membrane is connected to the shaft in a fluid-tight manner. If the membrane 400 encloses the first section of the shaft 401, a first interior space is formed between the membrane 400 and the shaft 401. The membrane 400 has at least one single perforation 402, which is formed in the variant depicted as a plurality of perforation lines. One of the plurality of perforation lines is marked by 410 and a dashed line. Each of the plurality of perforation lines is oriented along a longitudinal axis of the membrane 400. Each of the perforation lines begins at the centric periphery of the membrane 400 and ends in the variant depicted at the second end of the membrane 400.

Apart from the first electrode 403, two counter electrodes 404a, 404b are arranged on the shaft 401 outside the first interior space. The proximal end 420 of the shaft 401 is adapted to receive an electrically conductive fluid, which is formed as electrically conductive liquid in the variant depicted, and to convey it to the distal end 421 of the shaft 401. The first section of the shaft 401 is adapted to convey the electrically conductive liquid via the filling opening 405b in the wall of the shaft 401 into the first interior space. The first interior space is filled with the electrically conductive fluid and the first electrode 403 is flushed. The electrically conductive fluid emerges from the plurality of perforation lines. If an electrical signal is applied between the first electrode 403 and the counter electrode 404a, improved field line guidance takes place into the tissue to be ablated on account of the electrically conductive fluid that has emerged, which is explained in greater detail in FIG. 6.

A vent opening 405a and a filling opening 405b are arranged respectively on the first section of the membrane 400. If the device is held in a vertical position and the electrically conductive fluid conveyed via the filling opening into the first interior space, air can escape via the vent opening – the membrane 400 is vented.

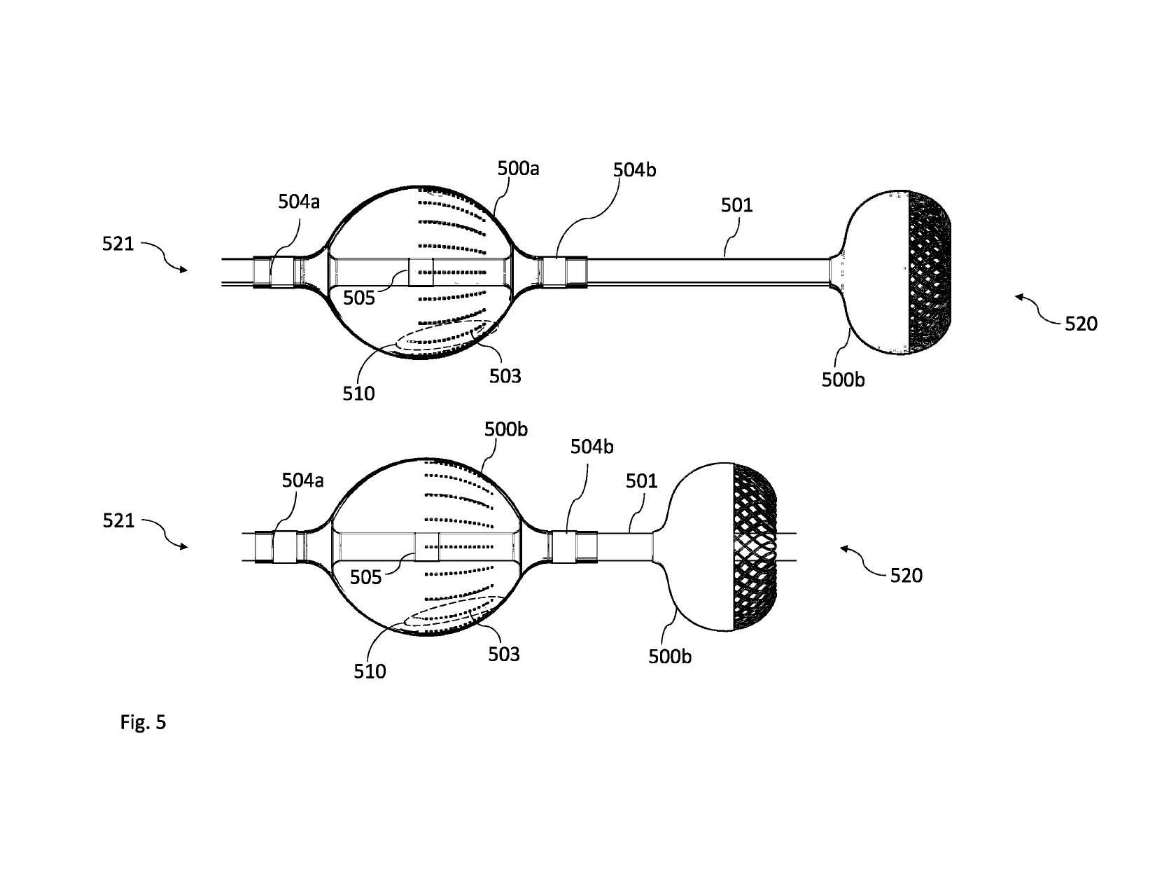

FIG. 5 shows a schematic representation of a variant of an embodiment with a containment element 500b.

The variant depicted has a shaft 501, which in turn has a proximal end 520 and a distal end 521. A first electrode 505 is arranged on a first section of the shaft 501. The first section is enclosed by a membrane 500a in a fluid-tight manner. Expressed differently, a first end and a second end of the membrane 500a are connected to the shaft 501 in a fluid-tight manner. If the membrane 500a encloses the first section of the shaft 501, a first interior space is formed between the membrane 500a and the shaft 501. The membrane 500a has at least one single perforation 503, which is formed in the variant depicted as a plurality of perforation lines. One of the plurality of perforation lines is marked by 510 and a dashed line. Each of the plurality of perforation lines is oriented along a longitudinal axis of the membrane 500a. Each of the perforation lines begins at the centric periphery of the membrane 500a and ends in the variant depicted at the first end of the membrane 500a.

Apart from the first electrode 505, two counter electrodes 504a, 504b are arranged on the shaft 501 outside the first interior space. The proximal end 520 of the shaft 501 is adapted to receive an electrically conductive fluid and to convey it to the distal end 521 of the shaft 501. The first section of the shaft 501 is adapted to convey the electrically conductive fluid into the first interior space. The first interior space is filled with the electrically conductive fluid. The electrically conductive fluid emerges from the plurality of perforation lines.

A containment structure 500b is arranged on a second section of the shaft 501.

In the variant depicted, when the device is guided into a lumen (not shown), an intermediate space is formed between the first membrane 500a, the containment structure 500b and the tissue wall (not shown) bordering the device. If the electrically conductive fluid emerges from the plurality of perforation lines, the intermediate space fills with the electrically conductive fluid and is in contact with the tissue wall. The containment structure can be displaced along a shaft axis, whereby it can be set which tissue surface amount of the lumen is in contact with the electrically conductive liquid.

The containment structure stops the electrically conductive fluid that has emerged from the plurality of perforation lines from spreading along a lumen axis when the intermediate space is filled with the fluid.

If an electrical signal is applied between the first electrode 505 and the counter electrode 504b, improved field line guidance takes place into the tissue to be ablated on account of the electrically conductive fluid that has emerged, which is explained in greater detail in FIG. 6.

FIG. 6 shows a schematic representation of a variant of an embodiment with a field pattern 511 during ablation. In the application depicted, the device from FIG. 5 was introduced into a tissue lumen G. The device is connected to a signal generator arrangement (not shown). The signal generator arrangement is connected to a control and evaluation unit (not shown). A fluid supply unit (not shown) is connected to the device and the control and evaluation unit and is adapted to provide an electrically conductive fluid.

The control and evaluation unit is adapted to set a volume flow of the fluid supply unit, in particular following a setting at the control and evaluation unit by a user. The first interior space thereupon fills with the electrically conductive fluid and it emerges from the plurality of perforation lines. The intermediate space, which is filled with the electrically conductive fluid, is represented by the hatching (diagonally dotted lines of a lumen wall to the opposing lumen wall) in FIG. 6. If an electrical signal is now applied between the first electrode 505 and the counter electrode 504b, a field line distribution is produced, of which one field line 511 is depicted as an example. On account of the direct contact of the electrically conductive fluid with the tissue G and the signal path merging via the electrically conductive fluid, a deeper penetration of the field lines into the tissue is achieved with simultaneous cooling of the tissue to be ablated.

Alternatively, the intermediate space can first be irrigated with a liquid active substance such as histamine. The histamine acts in the tissue and increases its electrical conductivity. Thereupon it can be set by a user at the control and evaluation unit that a second electrically conductive liquid is to be provided or should be provided by the fluid supply unit. The second electrically conductive liquid displaces the liquid active substance from the interior space of the membrane 500 and from the intermediate space.

If an electrical signal is now applied between the first electrode 505 and the counter electrode 504b, another field line distribution (not shown) results. On account of the direct contact of the second electrically conductive fluid with the tissue G and the signal path merging via the electrically conductive fluid, as well as the electrical conductivity of the tissue increased previously by the liquid active substance, a deeper penetration of the field lines into the tissue is achieved than without prior application of an active substance, with simultaneous cooling of the tissue to be ablated.

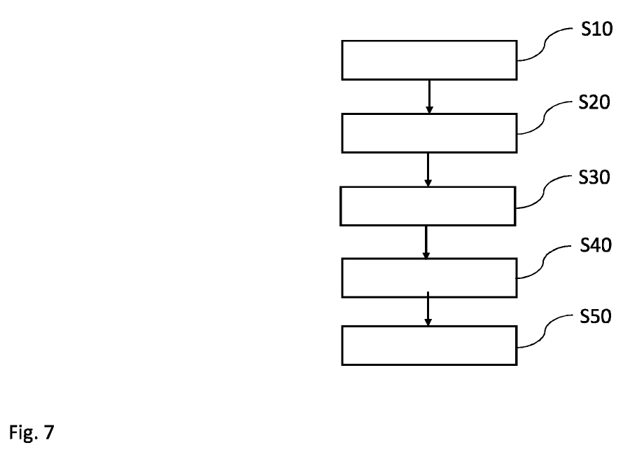

FIG. 7 shows a method for the delivery of electrical energy to tissue. The method comprises provision (step S10) of a system. The system has an application device, which is adapted to transmit an electrical signal to be received and a vasoactive, in particular liquid, substance to be received, in particular histamine, to/into the tissue. The system has a signal generator arrangement connected or connectable to the application device. The system has a control and evaluation unit connected or connectable to the application device and/or signal generator arrangement. The system has a fluid supply unit connected or connectable to the application device and/or control and evaluation unit, which fluid supply unit is adapted to provide the vasoactive substance. The method comprises contacting (step S20) of the application device with the tissue. The method comprises setting (step S30) of the control and evaluation unit to set the volume flow. The method comprises application (step S40) of the vasoactive substance. The method comprises activation (step S50) of the control and evaluation unit to generate an electrical signal.

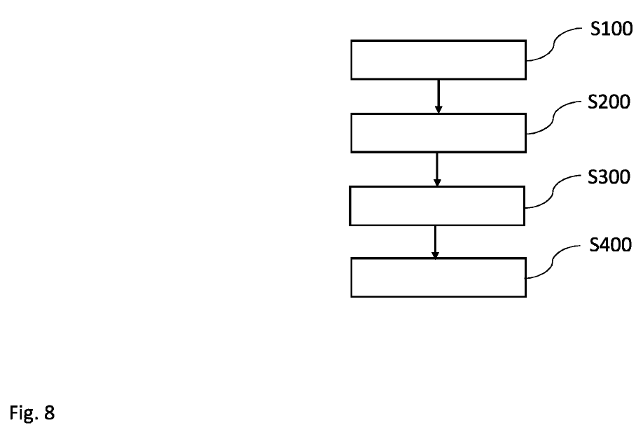

FIG. 8 shows a method for irreversible ablation, comprising the steps: provision (step S100) of a system having: a device as was described herein; a signal generator arrangement connected or connectable to the device, a control and evaluation unit connected or connectable to the device and/or signal generator arrangement, and a fluid supply unit connected or connectable to the device and/or control and evaluation unit, adapted to provide an electrically conductive fluid; introduction (step S200) of the device into a lumen of a patient as far as a tissue section to be ablated; setting (step S300) of a volume flow at the fluid supply unit; activation (step S400) of the control and evaluation unit to generate an electrical signal.

What is claimed is:

1. A catheter device for use in surgery, said device comprising:
a shaft, adapted to receive at least one electrically conductive fluid;
at least one first electrode arranged on a first section of the shaft;
at least one first counter electrode;
a first electric line connected to the at least one first electrode and the at least one first counter electrode, said first electric line being adapted to transmit an electrical signal to the at least one first electrode and the at least one first counter electrode; and
at least one first membrane adapted to enclose the first section of the shaft, to end with the shaft in a fluid-tight manner, and to form a first interior space between the at least one first membrane and the first section of the shaft, wherein the first section of the shaft is adapted to convey the at least one electrically conductive fluid into the first interior space, and wherein a first wall of the at least one first membrane is adapted to be at least partially fluid-permeable.

2. The device according to claim 1, wherein the at least one first membrane includes at least one first perforation and wherein the electrically conductive fluid can emerge from the at least one first perforation, and/or
the device further comprises:
at least one second electrode arranged on a second section of the shaft and connected to the first electric line; and/or
at least one second membrane including at least one second perforation, adapted to enclose the second section of the shaft, to end with the shaft in a fluid-tight manner, and to form a second interior space between the at least one second membrane and the second section of the shaft; and/or
wherein the second section of the shaft is adapted to convey the electrically conductive fluid into the second interior space, wherein a second wall of the at least one second membrane is adapted to be fluid-permeable, and/or
wherein the electrically conductive fluid emerges or can emerge from the at least one second perforation of the at least one second membrane, and/or
wherein the shaft comprises a distal end and a proximal end and the at least one first membrane is arranged between the distal end and the proximal end, and/or the at least one second membrane is arranged between the at least one first membrane and the proximal end.

3. The device according to claim 1, further comprising at least one containment structure that encloses a second part of the shaft in a fluid-tight manner, said at least one containment structure adapted to impede or confine the electrical conductive fluid emerging from the first wall and from the at least one first perforation in a propagation direction, and/or to be displaceable or displaced along the shaft, wherein the at least one containment structure is arranged between the at least one first membrane and the proximal end.

4. The device according to claim 3, further comprising a discharge opening arranged on the shaft between the at least one first membrane and the at least one containment structure or between the at least one first membrane and the at least one second membrane, said discharge opening being adapted to discharge the electrically conductive fluid that has emerged from the at least one first perforation, and/or from the at least one second perforation, wherein the shaft is adapted to convey the discharged electrically conductive fluid to the proximal end and out of the shaft; and/or
wherein at least one first and/or at least one second and/or at least one third counter electrode is arranged between the distal end and the at least one first membrane, or between the at least one first membrane and the proximal end, or between the at least one first membrane and the at least one second membrane, or between the at least one second membrane and the proximal end, or between the at least one first membrane and the at least one containment structure; and/or
wherein the at least one first and/or second and/or third counter electrode is formed as a body electrode.

5. The device according to claim 2, wherein the at least one first and/or the at least one second perforation is formed as:
at least one perforation line oriented along a longitudinal axis of the membrane starting from a middle/centric membrane periphery, in a longitudinal direction, towards a first or second end of the membrane; and/or
a plurality of perforation lines oriented along a longitudinal axis of the membrane starting from a middle/centric periphery, in a longitudinal direction, towards a first or second end of the membrane; and/or
a plurality of perforations arranged or distributed along or across a middle/centric periphery of the membrane.

6. The device according to claim 2, wherein the first and/or the second section include a filling opening, wherein the first and/or the second interior space is filled with the electrically conductive fluid via the proximal end.

7. The device according to claim 2, wherein the first and/or the second section include a vent opening, and/or the shaft includes a vent line arranged in the shaft and connected to the vent opening.

8. The device according to claim 2, wherein the first section and/or the second section and/or an exterior of the first and/or second membrane include a fluid pressure measurement sensor and/or a fluid flow measurement sensor, each adapted to provide measuring data, wherein the measuring data is read by a second electric line provided or arranged in the shaft.

9. The device according to claim 8, wherein the proximal end of the shaft includes an operating unit comprising:
an inflow and outflow connection adapted to convey an electrically conductive fluid to be received into the shaft to fill the interior spaces with electrically conductive fluid to be received, and to convey out of the shaft electrically conductive fluid that has emerged and been discharged; and/or
an electrical connection connected to the first and/or second electric line, said electrical connection adapted to transmit electrical signals to the first electric line and/or to transmit measuring data from the fluid pressure measurement sensor and/or fluid flow measurement sensor by the second electric line; and/or
an introducer opening adapted to receive a guide wire.

10. The device according to claim 2, wherein the first and/or second interior space include at least one first fluid chamber adapted to enclose the first and/or second section of the shaft in a fluid-tight manner and to convey the electrically conductive fluid to at least one partial surface of an interior surface of the first and/or second membrane.

11. The device according to claim 4, wherein the at least one first electrode, the at least one second electrode, the at least one first, second, third counter electrode, the at least one first membrane and/or the at least one second membrane, and the at least one first and/or second perforation include a bioresorbable material that is soluble in particular in bodily fluids, or is covered with a bioresorbable material that is soluble in particular in bodily fluids, or a corresponding material layer.

12. A system for the ablation of tissue, said system comprising:
at least one device according to claim 1;
at least one signal generator unit connected or connectable to the device;
at least one control and evaluation unit connected or connectable to the device and/or the signal generator unit, and
at least one fluid supply unit connected or connectable to the device and/or the control and evaluation unit, said at least one fluid supply unit being adapted to provide an electrically conductive fluid.

13. The system according to claim 12, wherein the control and evaluation unit is adapted to set a volume flow of the fluid supply unit, and wherein the fluid supply unit is adapted to provide the electrically conductive fluid according to the setting and to measure a fluid pressure and/or fluid flow by the fluid pressure measurement sensor and/or the fluid flow sensor.

14. The system according to claim 13, wherein the control and evaluation unit is adapted to determine and to output from the measured fluid pressure and/or fluid flow a measure for the positive fit between the membrane, in particular the at least one perforation, and the tissue to be ablated.

15. The system according to claim 12, wherein a first and/or second electrically conductive fluid is formed as an electrically conductive liquid and/or as an electrically conductive, liquid active substance, wherein the liquid or substance, upon contact with tissue, changes its electrical conductivity to increase or reduce it.

16. The system according to claim 12, wherein the signal generator unit includes a first signal generator for generating a radiofrequency signal and a second signal generator for generating a signal for pulsed field ablation.

17. The system according to claim 16, wherein the control and evaluation unit is adapted to activate the first signal generator and/or the second signal generator to emit a signal.

18. A method for the ablation of tissue, said method comprising:
providing a system comprising:
a device according to claim 1,
a signal generator arrangement connected or connectable to the device,
a control and evaluation unit connected or connectable to the device and/or the signal generator arrangement, and
a fluid supply unit connected or connectable to the device and/or the control and evaluation unit, adapted to provide an electrically conductive fluid;
introduction of the device into a lumen of a patient as far as a tissue section to be ablated;
setting a volume flow at the control and evaluation unit; and
activation of the control and evaluation unit to generate an electrical signal.

19. The method according to claim 18, wherein
the electrically conductive fluid comprises a vasoactive substance;
and/or comprising the steps:
fine-positioning of the device relative to the tissue section to be ablated; and/or
emission of an electrical signal; and/or
delivery of a vasoactive substance; and/or
removal of the device from the lumen of the patient.

20. A method for the delivery of electrical energy to tissue, said method comprising:
providing a system comprising:
an application device adapted to transmit an electrical signal to be received and a vasoactive substance to be received to/into the tissue,
a signal generator arrangement connected or connectable to the application device,
a control and evaluation unit connected or connectable to the application device and/or the signal generator arrangement, and
a fluid supply unit connected or connectable to the application device and/or the control and evaluation unit, said fluid supply unit adapted to provide the vasoactive substance;
contacting the application device to the tissue;
application of the vasoactive substance; and
activation of the control and evaluation unit to generate an electrical signal.

21. The method according to claim 20, further comprising:
setting of the control and evaluation unit to set the volume flow; and/or
following application, direct activation of the control and evaluation unit to generate an electrical signal.
